(19) United States
(12) Patent Application Publication
(10) Pub. No.: US 2026/0191751 A1
(43) Pub. Date: Jul. 9, 2026

(54) COSMETIC COMPOSITION FOR CARING KERATIN MATERIAL

(71) Applicant: L'OREAL, Paris (FR)

(72) Inventors: Yu HUANG, Shanghai (CN); Lingling SUN, Shanghai (CN)

(73) Assignee: L'OREAL, Paris (FR)

(21) Appl. No.: 19/132,610

(22) PCT Filed: Nov. 29, 2022
(86) PCT No.: PCT/CN2022/134987

§ 371 Date: May 23, 2025

(51) Int. Cl. A61K 8/365; A61K 8/34; A61K 8/368; A61K 8/86; A61Q 19/08

(52) U.S. Cl. CPC A61K 8/365; A61K 8/342; A61K 8/368; A61K 8/86; A61Q 19/08; A61K 2800/28

(57) ABSTRACT

A composition for caring keratin material, comprising: (i) at least one hydroxyl-containing acid, selected from the group consisting of α-hydroxyl acid, gluconic acid, lactobionic acid, and salicylic acid, or a precursor thereof; (ii) at least one short chain surfactant, each oleophilic chain of which contains 14 or less carbon atoms; (iii) at least two fatty alcohols having 20 or more carbon atoms; and (iv) at least one non-fatty alcohol surfactant other said short chain surfactant.

### TECHNICAL FIELD

The present invention relates to a cosmetic composition for caring keratin material, especially to a cosmetic composition comprising keratolytic agent(s).

### BACKGROUND ART

Skin renewal can be stimulated by removal of the outer keratinous layer. Such removal can be effected mechanically or chemically. Chemical peeling uses keratolytic agents that interact with the complex structure of the skin, removing the outer layer (stratum corneum for superficial peels, epidermis and possibly dermis for medium and deep peels) to induce a controlled injury to the skin and stimulate skin regeneration. The regenerated skin is smoother and appears younger.

“Keratolytic agent” refers to a natural or synthetic compound which has the effect of eliminating the dead cells of the stratum corneum, for example by stimulating the degradation corneodesmosine or stimulating renewal epidermal (including by stimulation of epidermal proliferation). Many current cosmetic products comprise such keratolytic agents to loosen and remove the stratum corneum of the users.

However, the keratolytic agent may lead to instability of the cosmetic product comprising the same, especially for the cosmetic product comprising a relatively higher amount of the keratolytic agent.

### SUMMARY OF THE INVENTION

The present invention aims at developing a new cosmetic product comprising at least one keratolytic agent. The cosmetic product remains stable at room temperature or at an elevated temperature, e.g. about 45° C., even when the amount of the keratolytic agent reaches about 5 wt. % or higher relative to the total weight of the cosmetic product while presenting no soaping effect.

According to the first aspect, the present invention relates to a composition for caring keratin material, comprising:
at least one hydroxyl-containing acid,
at least one short chain surfactant,
at least two fatty alcohols having 20 or more carbon atoms, and
at least one non-fatty alcohol surfactant other than said short chain surfactant.

The hydroxyl-containing acid is selected from the group consisting of α-hydroxyl acids, gluconic acid, lactobionic acid, salicylic acid, precursors thereof, and mixtures thereof. The expression “precursors thereof” means that the hydroxyl-containing acid may be chosen from the precursors of each of α-hydroxyl acids, gluconic acid, lactobionic acid, and salicylic acid.

Preferably, the α-hydroxyl acid is a lower acid having 2-12 carbon atoms, or 2-8 carbon atoms, or 2-6 carbon atoms.

In one embodiment, the composition can comprise a relatively higher amount of the hydroxyl-containing acid, e.g. an amount of 5 wt. % or more, or even an amount of 10 wt. % or more, relative to the total weight of the composition.

In one embodiment, the short chain surfactant means the surfactant each oleophilic chain of which contains 14 or less carbon atoms.

In one embodiment, the fatty alcohol is chosen from the saturated fatty alcohol having the formula of R1-OH, wherein the group R1 is a linear or branched alkyl, preferably a linear alkyl, with at least 20 carbon atoms.

Preferably, the composition comprises an amount of about 0.5 wt % or less of thickeners or gelling agents, and/or does not comprise any fatty alcohols having less than 20 carbon atoms, relative to the total weight of the composition.

The short chain surfactant is preferably chosen from polyoxyethylenated alkyl glycerides; fatty acid esters of glycerol or polyglycerol, which is optionally oxyalkylenated; oxyalkylenated fatty alcohol ethers; amino acid derivatives; betaines; and mixtures thereof.

The non-fatty alcohol surfactant other than said short chain surfactant can be chosen from glyceryl esters of fatty acids, polyethylene glycol esters of fatty acids, fatty acid esters of glucose or of alkylglucose, oxyethylenated ethers of a fatty acid and of glucose or of alkylglucose, alkylpolyglucosides, phospholipids, quaternary ammonium salts containing at least one hydrocarbyl having 16 or more carbon atoms, amino acid derivatives, and mixtures thereof.

The composition may comprise at least one emollient, which is preferably chosen from non-polar emollients and/or polar emollients, and the emollient can be present in an amount of about 5 wt. % or even higher, relative to the total weight of the composition.

Preferably, the polar emollient is selected from the group consisting of caprylic/capric triglyceride, isopropyl myristate, isopropyl palmitate, dibutyl adipate, propyleneglycol dicaprylate/dicaprate, cocoglyceride, cetearyl isononanoate, isopropyl myristate, isodecyl neopentanoate, tridecyl neopentanoate, and mixtures thereof.

Preferably, the non-polar emollient is selected from the group consisting of C₁₅₋₁₉ alkane, isohexadecane, isododecane, diethylhexyl cyclohexane, and mixtures thereof.

In one embodiment, the composition further comprises water, and the water is present in an amount ranging from about 20 wt. % to about 90 wt. %, relative to the total weight of the composition.

Further, the present invention relates to a composition for caring keratin material, comprising a keratolytic system, a liquid crystal system and at least one emollient, wherein the keratolytic system comprises at least one hydroxyl-containing acid, at least one short chain surfactant, or mixtures thereof, and the liquid crystal system comprises, consists essentially of, or consists of at least two fatty alcohols having 20 or more carbon atoms and at least one non-fatty alcohol surfactant other than said short chain surfactant, wherein the hydroxyl-containing acid, the short chain surfactant, the fatty alcohols having or more carbon atoms, the non-fatty alcohol surfactant other than said short chain surfactant and the emollient are defined as above.

According to the second aspect, the present invention relates to use of a liquid crystal system for improving stability of a composition comprising a keratolytic system and optionally at least one emollient, wherein the keratolytic system comprises at least one hydroxyl-containing acid and at least one short chain surfactant, and wherein the liquid crystal system comprises, consists essentially of, or even consists of at least two fatty alcohols having 20 or more carbon atoms and at least one non-fatty alcohol surfactant other than said short chain surfactant.

In one embodiment, the composition comprises an amount of about 0.5 wt % or less of thickeners or gelling agents and/or does not comprise any fatty alcohols having less than 20 carbon atoms, relative to the total weight of the composition.

In one embodiment, the composition can comprise a relatively higher amount of the hydroxyl-containing acid, e.g. an amount of about 5 wt. % or higher, or even an amount of about 10 wt. % or more, relative to the total weight of the composition, and/or the composition can comprise a relatively higher amount of the emollient, e.g. an amount of about 5 wt. % or higher, relative to the total weight of the composition.

Other subjects and characteristics, aspects and advantages of the present invention will emerge even more clearly on reading the detailed description and the examples that follow.

### DETAILED DESCRIPTION OF THE INVENTION

In that which follows and unless otherwise indicated, the limits of a range of values are included within this range, in particular in the expressions “of between” and “ranging from . . . to . . . ”.

The articles “a” and “an”, as used herein, mean one or more when applied to any feature in embodiments of the present invention described in the specification and claims. The use of “a” and “an” does not limit the meaning to a single feature unless such a limit is specifically stated. Moreover, the expression “at least one” used in the present description is equivalent to the expression “one or more”.

Throughout the present application, the expression “comprising” is to be interpreted as encompassing all specifically mentioned features as well optional, additional, unspecified ones. As used herein, the use of the term “comprising” also discloses the embodiment wherein no material features or even no features other than the specifically mentioned features are present (such as “consisting essentially of” and “consisting of”). In the case of “consisting essentially of,” any additional compositions, materials, and/or components that materially affect the basic and novel characteristics are excluded from such an embodiment, but any compositions, materials and/or components that do not materially affect the basic and novel characteristics can be included in the embodiment.

The term “about” denoting a certain value is intended to denote a range within +5% of the value. As one example, the phrase “about 100” denotes a range of 100±5, i.e. the range from 95 to 105. Generally, when the term “about” is used, it can be expected that similar results or effects according to the disclosure can be obtained within a range of +5% of the indicated value.

As used herein, the term “keratin material” is understood to mean the skin, the nails, or the mucous membranes, and, in at least one embodiment, refers to the skin.

### Keratolytic System

The composition of the present invention may comprise a keratolytic system comprising at least one hydroxyl-containing acid, at least one short chain surfactant, or mixtures thereof.

### Hydroxyl-Containing Acids

The composition of the present invention comprises at least one hydroxyl-containing acid.

According to the present invention, the hydroxyl-containing acid can be particularly an α-hydroxyl acid. The term “α-hydroxyl acid” is understood to mean a carboxylic acid having at least one hydroxyl functional group occupying an α-position on said acid (carbon adjacent to a carboxylic acid functional group). This acid is preferably present in the final composition in the form of the free acid.

The α-hydroxyl acids (α-hydroxyl acid) include, for example, citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid and their mixtures. It is also possible to use mixtures of these various acids.

In one embodiment, the α-hydroxyl acid is preferably a lower acid having 2-12 carbon atoms, or 2-8 carbon atoms, or 2-6 carbon atoms.

In one embodiment, the α-hydroxyl acid is preferably a mono-carboxylic acid.

Examples of the hydroxyl-containing acid include, but are not limited to, glycolic acid, lactic acid, malic acid, citric acid, gluconic acid, lactobionic acid, tartaric acid and salicylic acid.

In one embodiment, a precursor of a hydroxyl-containing acid can also be used for the present composition. According to the present invention, a “precursor” means one capable of being converted into a hydroxyl-containing acid useful for the invention, e.g., through hydrolysis. The example of such a precursor can be particularly made to gluconolactone. For the purpose of the invention, especially considering the composition according to the present invention being aqueous, such a precursor can be deemed as belonging to the conception of “hydroxyl-containing acid”.

Examples of the precursors of a hydroxyl-containing acid may be chosen from lactone precursors, which include, but are not limited to, erythronolactone, threolactone, ribonolactone, arabinolactone, xyloslactone, lyxoslactone, allolactone, altrolacone, gluconolactone, mannolactone, gulonolactone, idolactone, galactonolactone, talolactone, and mixtures thereof.

In one embodiment, the keratolytic system comprises one or more α-hydroxy-acid(s), e.g. lactic acid; salicylic acid; one or more precursor(s) of the hydroxyl-containing acid(s), e.g. gluconolactone, or mixtures thereof.

According to the present invention, the hydroxyl-containing acid is present in an amount ranging from about 0.5 wt. % to about 30 wt. %, from about 1 wt. % to about 25 wt. %, from about 2 wt. % to about 20 wt. %, or from about 5 wt. % to about 15 wt. %, e.g. about 8 wt. % to about 12 wt. %, relative to the total weight of the composition.

### Short Chain Surfactants

Conventionally, some hydroxyl-containing acids can serve as keratolytic agents, e.g. those as mentioned above. Surprisingly, the inventor discovers that some short chain surfactants can also play a role of keratolytic agents.

The composition of the present invention comprises at least one short chain surfactant.

According to the present invention, the term “short chain surfactant” means the surfactant each oleophilic chain of which contains 14 or less carbon atoms. According to the present invention, the short chain surfactant can be comprised in the composition as a mixture, wherein the mixture comprises at least about 40 wt. %, at least about 50 wt. %, or even at least about 60 wt. % of one or more said short chain surfactant(s), e.g. of two or three said short chain surfactant(s), based on the total weight of the mixture.

Further, the short chain surfactant is preferably chosen from anionic surfactants, amphoteric surfactants, nonionic surfactants or mixtures thereof. More preferably, the short chain surfactant is chosen from nonionic surfactants.

Examples of the anionic surfactant include, but are not limited to, amino acid derivatives, such as sodium lauroyl sarcosinate, sodium methyl cocoyl taurate, disodium lauroyl glutamate, sodium caproyl aspartate, sodium cocoyl alaninate, and mixtures thereof.

Examples of the amphoteric surfactant include, but are not limited to, betaines, such as cocamidopropyl betaine, lauramidopropyl betaine, capryl/capramidopropyl betaine, coco betaine, and mixtures t and amphodiacetates, e.g. disodium cocoamphodiacetate.

The nonionic surfactant is preferably chosen from PEG/PG-derived short chain surfactants. According to the present invention, the term “PEG/PG-derived short chain surfactant” means the surfactant which is derived from polyethylene glycols and/or polyglycerols and each oleophilic chain of which contains 14 or less carbon atoms.

Non-limiting examples of the PEG/PG-derived short chain surfactants useful in the present invention include, for example, polyoxyethylenated alkyl glycerides; fatty acid esters of glycerol or polyglycerol, which is optionally oxyalkylenated; oxyalkylenated fatty alcohol ethers; or mixtures thereof.

Polyoxyethylenated alkyl glycerides are preferably those including polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units), e.g., PEG-6 Caprylic/Capric Glycerides, and PEG-7 Caprylic/Capric Glycerides.

In the context of the present invention, mention may also be made of oxyalkylenated fatty acid esters of glycerol and in particular polyoxyethylenated derivatives of glyceryl esters of fatty acids and hydrogenated derivatives thereof. These oxyalkylenated glycerol esters can be chosen, for example, from glyceryl esters of fatty acids which are hydrogenated and/or oxyethylenated, such as oxyethylenated glyceryl cocoates, such as PEG-7 glyceryl cocoate, PEG-30 glyceryl cocoate, and mixtures thereof.

In one embodiment, the fatty acid ester of polyglycerol is chosen from polyglyceryl monolaurate comprising from 1 to 20, preferably from 2 to 10 glycerol units, polyglyceryl monomyristate comprising from 1 to 20, preferably from 2 to 10 glycerol units, polyglyceryl monolaurate comprising from 1 to 20, preferably from 2 to 10 glycerol units, polyglyceryl monocaprate comprising from 1 to 20, preferably from 2 to 10 glycerol units, polyglyceryl monocaprylate comprising from 1 to 20, preferably from 2 to 10 glycerol units, and mixtures thereof.

Examples of the fatty acid ester of polyglycerol may include, but not be limited to polyglyceryl-2 laurate, polyglyceryl-3 laurate, polyglyceryl-4 laurate, polyglyceryl-5 laurate, polyglyceryl-6 laurate, polyglyceryl-10 laurate, polyglyceryl-2 myristate, polyglyceryl-3 myristate, polyglyceryl-4 myristate, polyglyceryl-5 myristate, polyglyceryl-6 myristate, polyglyceryl-10 myristate, polyglyceryl-4 caprylate/caprate, polyglyceryl-10 caprylate/caprate, polyglyceryl-2 caprate, polyglyceryl-4 caprate, polyglyceryl-10 caprate, and polyglyceryl-10 cocoate.

Oxyalkylenated (oxyethylenated and/or oxypropylenated) fatty alcohol ethers are preferably those having a number of ethylene or propylene oxide (EO/PO) units ranging from 2 to 40. Mention may be made to PEG-4 lauryl ether, PEG-9 lauryl ether and PEG-23 lauryl ether.

Preferably, the PEG/PG-derived short chain surfactants are chosen from fatty acid esters of polyglycerol, e.g. polyglyceryl-5 laurate, oxyethylenated fatty acid esters of glycerol, e.g. PEG-7 glyceryl cocoate, and polyoxyethylenated alkyl glycerides, e.g. PEG-6 Caprylic/Capric Glycerides, or mixtures thereof.

According to the present invention, the short chain surfactant is present in an amount ranging from about 0.1 wt. % to about 15 wt. %, preferably from about 0.5 wt. % to about 10 wt. %, or from about 1 wt. % to about 5 wt. %, relative to the total weight of the composition.

Preferably, the keratolytic system comprises both hydroxyl-containing acids and PEG/PG derived short chain surfactants as defined above. In one embodiment, the keratolytic system comprises one or more of glycolic acid, lactic acid, gluconolactone, and salicylic acid; and one or more of fatty acid ester of polyglycerol, e.g. polyglyceryl-5 laurate, oxyethylenated fatty acid esters of glycerol, e.g. PEG-7 glyceryl cocoate, and polyoxyethylenated alkyl glycerides, e.g. PEG-6 Caprylic/Capric Glycerides.

According to the present invention, the keratolytic system is present in an amount ranging from about 0.5 wt. % to about 45 wt. %, preferably from about 1 wt. % to about 30 wt. %, or from about 2 wt. % to about 20 wt. %, relative to the total weight of the composition.

### Liquid Crystal System

The composition of the present invention preferably comprises a liquid crystal system, which comprises at least two fatty alcohols and at least one main surfactant which is different from the above short chain surfactant.

### Fatty Alcohols

The composition of the present invention comprises at least two fatty alcohols having 20 or more carbon atoms.

The fatty alcohol may be the saturated fatty alcohol of formula (I):

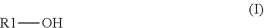

wherein R1 is a linear or branched alkyl with at least 20 carbon atoms.

Preferably, in formula (I), the group R1 is a linear or branched alkyl with 20-40 carbon atoms, preferably 20-30 carbon atoms, and more preferably 20-24 carbon atoms.

As examples of saturated fatty alcohol of formula (I), arachidyl alcohol, behenyl alcohol, lignoceryl alcohol (1-tetracosanol), ceryl alcohol (1-hexacosanol), montanyl alcohol (1-octacosanol), and myricylic alcohol (1-triacontanol) can be mentioned.

In a preferred embodiment, the composition of the present invention comprises a mixture of saturated fatty alcohols of formula (I) with 20-22 carbon atoms. For example, the composition of the present invention comprises a mixture of arachidyl alcohol and behenyl alcohol.

As an example of saturated fatty alcohol of formula (I) useful in the composition according to the present invention, the commercial product, Nafol 1822 C from Condea, can be cited. Nafol 1822 C is referenced as behenyl alcohol and is defined to be a mixture of fatty alcohols containing chiefly n-docosanol, a C₂₂ alcohol. Another commercial product that can be used in the composition of the present invention is Lannet 22 from COGNIS (BASF) that contains behenyl alcohol, and Lanette 22 from BASF. Other representative mixtures of fatty alcohols are the product sold under the name Nafol 2298 by the company Condea, which contains 98% C₂₂ alcohol, and C₂₀₋₂₂ ALCOHOLS under the trade name of NAFOL 2022 EN from SASOL.

In one embodiment, the fatty alcohols are chosen from the saturated fatty alcohol of formula (I) wherein R1 is a linear alkyl chain with 20-40 carbon atoms, preferably 20-30 carbon atoms, and more preferably 20-24 carbon atoms.

According to the present invention, the fatty alcohols having 20 or more carbon atoms are present in an amount ranging from about 0.1 wt. % to about 10 wt. %, preferably from about 0.5 wt. % to about 8 wt. %, or from about 1 wt. % to about 5 wt. %, relative to the total weight of the composition.

### Main Surfactants

The composition of the present invention comprises at least one non-fatty alcohol surfactant other than said short chain surfactant as the main surfactant. The expression “non-fatty alcohol surfactant other than said short chain surfactant” means a surfactant which is not a fatty alcohol and is different from the short chain surfactant as defined above.

The non-fatty alcohol surfactant other than said short chain surfactant is preferably chosen from nonionic surfactants, anionic surfactants, cationic surfactant, amphoteric surfactants, or mixtures thereof. More preferably, the non-fatty alcohol surfactant other than said short chain surfactant is chosen from nonionic surfactants, amphoteric surfactant or mixtures thereof.

Examples of the anionic surfactant include, but are not limited to, amino acid derivatives, such as sodium methyl stearoyl taurate, disodium stearoyl glutamate, sodium methyl oleoyl taurate, sodium palmitoyl glutamate, and mixtures thereof.

Examples of the cationic surfactant include, but are not limited to, quaternary ammonium salts containing at least one hydrocarbyl having 16 or more carbon atoms, such as stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and mixtures thereof.

Examples that may be mentioned for the nonionic surfactant include esters of polyols and of fatty acids with a saturated or unsaturated chain containing, for example, from 8 to 24 carbon atoms and better still from 12 to 22 carbon atoms, such as the glyceryl esters of C₈-C₂₄ fatty acids; the polyethylene glycol esters of C₈-C₂₄ fatty acids; the sugar (sucrose, glucose or alkylglucose) esters of C₈-C₂₄ fatty acids, and the oxyalkylenated derivatives thereof; the sugar ethers of C₈-C₂₄ fatty alcohols; and mixtures thereof.

Glyceryl esters of fatty acids that may especially be mentioned include glyceryl stearate (glyceryl monostearate, distearate and/or tristearate) (CTFA name: glyceryl stearate) or glyceryl ricinoleate, and mixtures thereof.

Polyethylene glycol esters of fatty acids that may especially be mentioned include polyethylene glycol stearate (polyethylene glycol monostearate, distearate and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

Mixtures of these surfactants may also be used, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (CTFA name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: methyl glucose dioleate); the mixed ester of methylglucoside and of the oleic acid/hydroxystearic acid mixture (CTFA name: methyl glucose dioleate/hydroxysterate); the ester of methylglucoside and of isostearic acid (CTFA name: methyl glucose isostearate); the ester of methylglucoside and of lauric acid (CTFA name: methyl glucose laurate); the mixture of the monoester and diester of methylglucoside and of isostearic acid (CTFA name: methyl glucose sesquiisostearate); the mixture of the monoester and diester of methylglucoside and of stearic acid (CTFA name: methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate.

Sugar ethers that may especially be mentioned are alkylpolyglucosides, for example decylglucoside, for instance the product sold under the name Mydol 10 by the company Kao Chemicals, the product sold under the name Plantaren 2000 by the company Henkel, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Henkel; arachidyl glucoside, for example in the form of the mixture of arachidyl alcohol and behenyl alcohol and arachidyl glucoside, sold under the name Montanov 202 by the company SEPPIC; cocoylethylglucoside, for example in the form of the mixture (35/65) with cetyl alcohol and stearyl alcohol, sold under the name Montanov 82 by the company SEPPIC; and mixtures thereof.

Examples of the amphoteric surfactant, include, but are not limited to, phospholipids that may be mentioned include phosphatidic acid (phosphatidate), phosphatidylethanolamine (cephalin), phosphatidylcholine (lecithin of any origin (soya, sunflower or marine)), hydrogenated lecithin, phosphatidylserine, phosphatidylinositol phosphate, phosphatidylinositol bisphosphate, phosphatidylinositol triphosphate, ceramide phosphorylcholine (Sphingomyelin), ceramide phosphorylethanolamine (Sphingomyelin), ceramide phosphorylglycerol and mixtures thereof.

Other surfactants, as the main surfactants, may include sorbitan monoesters like sorbitan stearate, sorbitan tristearate, sorbitan palmitate, sorbitan laurate, cholesterol, lanolin, and phytosterols.

Preferably, the non-fatty alcohol surfactant other than said short chain surfactant is a non-ionic surfactant, an amphoteric surfactant, or mixtures thereof,

Preferably, the non-fatty alcohol surfactant other than said short chain surfactant is chosen from glyceryl esters of fatty acids, polyethylene glycol esters of fatty acids, fatty acid esters of glucose or of alkylglucose, oxyethylenated ethers of a fatty acid and of glucose or of alkylglucose, alkylpolyglucosides, phospholipids and mixtures thereof, more preferably chosen from alkylpolyglucosides, phospholipids or mixtures thereof, more preferably chosen from decylglucoside, laurylglucoside, cocoglucoside, cetostearyl glucoside, arachidyl glucoside, hydrogenated lecithin, and mixtures thereof.

According to the present invention, the non-fatty alcohol surfactant other than said short chain surfactant is present in an amount ranging from about 0.05 wt. % to about 10 wt. %, preferably from about 0.08 wt. % to about 5 wt. %, or from about 0.1 wt. % to about 2 wt. %, relative to the total weight of the composition.

### Emollients

The composition of the present invention may comprise at least one emollient. The emollient may be a polar emollient and/or a non-polar emollient.

As used herein, the term “polar emollient” means any emollient having at least one polar moiety. The emollient may be one or both of high and medium polarity oil soluble emollients.

Non-limiting examples of polar emollients include, but are not limited to, esters, polyol esters, and polyol ethers such as linear or branched polyglycerol esters and polyglycerol ethers. For example, the polar emollient can be chosen from caprylic/capric triglyceride, isopropyl myristate, PPG-3 myristyl ether, isopropyl palmitate, dibutyl adipate, propyleneglycol dicaprylate/dicaprate, cocoglyceride, cetearyl isononanoate, isopropyl myristate, isodecyl neopentanoate, tridecyl neopentanoate, C₁₂₋₁₅ alkyl benzoate, phenethyl benzoate, and mixtures thereof. Other examples of emollients include oil soluble emollients having high or medium polarity moieties.

The emollient may be a non-polar oil soluble emollient. The term “non-polar emollient” means any emollient possessing no permanent electric moments.

Non-limiting examples of non-polar emollients may include, but are not limited to, non-polar hydrocarbons, such as linear or branched hydrocarbons. For example, the emollients may be chosen from paraffins, isoparaffins, mineral oil, C₁₅₋₁₉ alkane, isohexadecane, isododecane, diethylhexyl cyclohexane, and mixtures thereof. In some instances, the non-polar emollient is chosen from isododecane, C₁₅₋₁₉ alkane and mixtures thereof.

According to the present invention, the emollient is present in an amount ranging from about 0.1 wt. % to about 20 wt. %, preferably from about 1 wt. % to about 15 wt. %, or from about 5 wt. % to about 10 wt. %, relative to the total weight of the composition.

### Solvent

Generally, the composition of the present invention comprises at least one solvent. The solvent of the composition of the present invention may include one or more water-miscible or at least partially water-miscible compounds (at room temperature of 20-25° C.), for instance C₂-C₈ lower polyols, monoalcohols, or polyol ethers (especially containing from 3 to 16 carbon atoms). The solvent of the composition of the present invention may include water, or consist essentially of water.

The term “polyol” should be understood as meaning any organic molecule comprising at least two free hydroxyl groups. Examples of polyols that may be mentioned include glycols, for instance butylene glycol, propylene glycol, pentylene glycol, isoprene glycol, caprylyl glycol, glycerol (i.e. glycerin) and polyethylene glycols.

The monoalcohol may be chosen from ethanol, isopropanol and mixtures thereof.

The polyol ether may be chosen from mono-, di- or tripropylene glycol (C₁-C₄)alkyl ethers, mono-, di- or triethylene glycol (C₁-C₄)alkyl ethers, and combinations thereof.

Advantageously, the solvent is present in an amount ranging from about 20 wt. % to about 90 wt. %, preferably from about 30 wt. % to about 85 wt. %, or from about 40 wt. % to about 80 wt. %, relative to the total weight of the composition.

### Other Ingredients

The composition according to the present invention may also comprise other ingredients, known previously elsewhere in cosmetic compositions, such as cosmetic active ingredients, thickeners or gelling agents, sequestering agents, preserving agents, fragrances, and so on.

In particular, the cosmetic active agent may be at least one hydrophilic active agent. The term “hydrophilic active agent” means a water-soluble or water-dispersible active agent that is capable of forming hydrogen bonds.

As hydrophilic active agents, examples that may be mentioned include moisturizers, depigmenting agents, humectants, anti-ageing agents; mattifying agents, cicatrizing agents, antibacterial agents, and mixtures thereof.

The hydrophilic active agent(s) may notably be chosen from:
vitamins and derivatives thereof, notably esters thereof, such as tocopherol (vitamin E) and esters thereof (for instance tocopheryl acetate), and ascorbic acid or a derivative thereof;
humectants,
C-glycoside compounds, preferably hydroxypropyl tetrahydropyrantriol;
antioxidant compounds;
anti-ageing active agents, such as hyaluronic acid compounds, and notably sodium hyaluronate, salicylic acid compounds and in particular 5-n-octanoylsalicylic acid (capryloylsalicylic acid), adenosine, C-β-D-xylopyranoside-2-hydroxypropane and the sodium salt of (3-hydroxy-2-pentylcyclopentyl) acetic acid; and
mixtures thereof.

Advantageously, the hydrophilic active agent is present in an amount ranging from 0 wt. % to about 40 wt. %, e.g. about 10 wt. % to about 30 wt. %, relative to the total weight of the composition.

Advantageously, the thickener or gelling agent is present in an amount ranging from 0 wt. % to about 0.5 wt. %, relative to the total weight of the composition.

### Compositions

As understood in the art, it is difficult to stabilize the composition comprising a short chain surfactant and a relatively higher amount of a hydroxyl-containing acid.

By means of the above liquid crystal system, the composition which further comprises a short chain surfactant and a relatively higher amount of a hydroxyl-containing acid can achieve good stability even at an elevated temperature, e.g. about 45° C.

In one embodiment, the composition of the present invention may comprise one or more fatty alcohols having less than 20 carbon atoms. Preferably, the composition of the present invention does not comprise any fatty alcohols having less than 20 carbon atoms.

In one embodiment, the ratio of the above fatty alcohols plus the above main surfactants to the emollients by weight is about 10 or higher, preferably about 15 or higher, or about 18 or higher, e.g. about 18.7% or higher.

In one embodiment, the ratio of the above main surfactants to the above fatty alcohols by weight is about 1/10 or higher, preferably about ⅛ or higher, or about ⅙ or higher.

By means of the specific ratios, the composition of the present invention achieves improved stability and good skin sensation e.g. no soaping when being applied on skins.

### Method and Use

According to another aspect, the present invention relates to a process for caring keratin materials, especially skins, by applying the composition according the present invention on the keratin material.

In one embodiment, the process comprises the following steps in order, washing the skin, wiping the skin, and applying the composition according the present invention on the skin, and optionally massaging the skin for about 2 seconds to about 40 seconds.

According to still another aspect, the present invention relates to use of a liquid crystal system for improving stability of a composition comprising a keratolytic system and optionally at least one emollient.

In one embodiment, the keratolytic system comprises at least one hydroxyl-containing acid and at least one short chain surfactant, and the liquid crystal system comprises, consists essentially of, or even consists of at least two fatty alcohols having 20 or more carbon atoms and at least one non-fatty alcohol surfactant other than said short chain surfactant.

The present invention is illustrated in greater detail by the examples described below, which are given as non-limiting illustrations.

### EXAMPLES

Main raw materials used, trade names and supplier thereof were listed in Table 1.

TABLE 1

| INCI Name | Trade Name | Supplier |
|---|---|---|
| HYDROGENATED | NIKKOL LECINOL | NIKKO |
| LECITHIN | S 10 | |
| BEHENYL ALCOHOL | LANETTE 22 | BASF |
| C20-22 ALCOHOLS | NAFOL 2022 EN | SASOL |
| LACTIC ACID | L-LACTIC ACID | MUSASHINO |
| | (90%)(CRG) | CHEMICAL |
| | | LABORATORY |
| SALICYLIC ACID | AMIPRESERVE | CRODA |
| PEG-7 GLYCERYL | CETIOL HE | BASF |
| COCOATE | | |
| POLYGLYCERYL-5 | SUNSOFT A- | TAIYO KAGAKU |
| LAURATE | 121E-C | |
| GLUCONOLACTONE | GLUCONO- | JUNGBUNZLAUER |
| | DELTA- | |
| | LACTONE F5010 | |
| | PERSONAL CAR | |
| CETEARYL | CETIOL SN | BASF |
| ISONONANOATE | | |
| C₁₅₋₁₉ ALKANE | EMOGREEN L19 | SEPPIC |
| ISOHEXADECANE | ISOHEXADECANE | INEOS |
| ARACHIDYL | MONTANOV 202 | SEPPIC |
| ALCOHOL | | |
| (and) | | |
| BEHENYL | | |
| ALCOHOL | | |
| (and) | | |
| ARACHIDYL | | |
| GLUCOSIDE | | |
| CETEARYL ALCOHOL | LANETTE O OR | BASF |
| STEARYL ALCOHOL | LANETTE 18 | BASF |

### Inventive Examples 1-2 and Comparative Examples 1-3

The compositions according to inventive formulas Exs. 1-2 and comparative formulas CEs. 1-3 were prepared mainly with the ingredients listed in Table 2 (the contents were expressed as weight percentages of ingredients with regard to the total weight of each composition, unless otherwise indicated):

TABLE 2

| Components | Ex. 1 | Ex. 2 | CE. 1 | CE. 2 | CE. 3 |
|---|---|---|---|---|---|
| HYDROGENATED | 0.3 | | 0.3 | 0.3 | 0.3 |
| LECITHIN | | | | | |
| ARACHIDYL ALCOHOL | | 1.8 | | | |
| (and) BEHENYL | | | | | |
| ALCOHOL (and) | | | | | |
| ARACHIDYL | | | | | |
| GLUCOSIDE | | | | | |
| STEARYL ALCOHOL | | | | | 0.75 |
| CETEARYL ALCOHOL | | | 1.5 | | |
| BEHENYL ALCOHOL | 0.37 | | | 1.5 | 0.75 |
| C₂₀₋₂₂ ALCOHOLS | 1.25 | | | | |
| LACTIC ACID | 6.66 | 6.66 | 6.66 | 6.66 | 6.66 |
| SALICYLIC ACID | 0.2 | 0.2 | 0.2 | 0.2 | 0.2 |
| GLUCONOLACTONE | 5 | 5 | 5 | 5 | 5 |
| PEG-7 GLYCERYL | 0.5 | 0.5 | 0.5 | 0.5 | 0.5 |
| COCOATE | | | | | |
| POLYGLYCERYL-5 | 1 | 1 | 1 | 1 | 1 |
| LAURATE | | | | | |
| CETEARYL | 2 | 2 | 2 | 2 | 2 |
| ISONONANOATE | | | | | |
| ISOHEXADECANE | 2 | 2 | 2 | 2 | 2 |
| C₁₅₋₁₉ ALKANE | 4 | 4 | 4 | 4 | 4 |
| WATER | QS | QS | QS | QS | QS |

Note:
ARACHIDYL ALCOHOL (and) BEHENYL ALCOHOL (and) ARACHIDYL GLUCOSIDE: 55% ARACHIDYL ALCOHOL; 30 wt. % BEHENYL ALCOHOL; 15 wt. % ARACHIDYL GLUCOSIDE.

### Preparation Procedure:

The compositions were prepared as follows:
1). adding water, the alcohol(s), and each of the surfactant(s) into the main kettle and heating the mixture to 80° C.;
2). gradually adding the emollients into the main kettle with strong homogenization;
3). after homogenization for 5 min, cooling the mixture and keeping it homogeneous;
4). weakening the homogenization when the temperature decreased to 45° C.; and
5). adding other ingredients, if present, and cooling the mixture to 30° C. and discharging the compositions.

### Shaking Stability Test

Stability property was evaluated according to the following process:
1) adding the compositions into 60 ml glass stability bottles;
2) putting the bottles in the ovens of 4° C., 37° C., and 45° C. for 2 month, respectively; and
3) evaluating the appearance, color and odor of the compositions after 2 month.

### Soaping Effect

Normal amounts of the compositions were applied on the backs of the users' hands and the appearing white trace during application was checked, wherein no white trace or slight white trace was acceptable, otherwise was not acceptable.

TABLE 3
Performance of the compositions

| Properties | Ex. 1 | Ex. 2 | CE. 1 | CE. 2 | CE. 3 |
|---|---|---|---|---|---|
| Soaping | No | No | Soaping | Soaping | Soaping |
| effect | soaping | soaping | | | |
| Stability | Passed | Passed | Phase | Phase | Not stable, |
| | | | separation | separation | got thinner |
| | | | at T0 | at 45° C. | at 45° C. |
| | | | | after 2 | |
| | | | | month | |

From the data in Table 3, it could be seen that, at relatively higher amounts of each of keratolytic agents, only the compositions comprising at least two fatty alcohols having 20 or more carbon atoms and a main surfactant could achieve the desirable stability.

By contrast, even for the compositions comprising one fatty alcohol having 20 or more carbon atoms and one fatty alcohol having less than 20 carbon atoms, desirable 5 stability cannot be achieved, as shown by the effects of Comparative Example 3.

Further, only the compositions comprising at least two fatty alcohols having 20 or more carbon atoms and a main surfactant could achieve better skin sensation e.g. no soaping. However, the compositions which only comprised one fatty alcohol having 20 or more carbon atoms or the compositions which did not comprise any fatty alcohols having 20 or more carbon atoms cannot achieve desirable skin sensation since these compositions led to soaping when being applied on the skins.

1. A composition for caring keratin material, comprising:
(i) a hydroxyl-containing acid, selected from the group consisting of α-hydroxyl acids, gluconic acid, lactobionic acid, salicylic acid, precursors thereof, and mixtures thereof;
(ii) a short chain surfactant, each oleophilic chain of which contains 14 or less carbon atoms;
(iii) at least two fatty alcohols having 20 or more carbon atoms; and
(iv) a non-fatty alcohol surfactant different from the short chain surfactant.

2. The composition according to claim 1, wherein the α-hydroxyl acid is a lower acid having 2-12 carbon atoms.

3. The composition according to claim 1, wherein the hydroxyl-containing acid is selected from the group consisting of glycolic acid, lactic acid, salicylic acid, gluconolactone, and mixtures thereof.

4. The composition according to claim 1, wherein the fatty alcohol is a saturated fatty alcohol of formula (I): 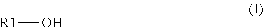
wherein the group R1 is a linear or branched alkyl with at least 20 carbon atoms.

5. The composition according to claim 1, wherein the composition does not comprise a fatty alcohol having less than 20 carbon atoms.

6. The composition according to claim 1, wherein the short chain surfactant is derived from polyethylene glycols and/or polyglycerols.

7. The composition according to claim 1, wherein the short chain surfactant is selected from the group consisting of polyoxyethylenated alkyl glycerides; fatty acid esters of glycerol, optionally oxyalkylenated, fatty acid esters of polyglycerol, optionally oxyalkylenated; oxyalkylenated fatty alcohol ethers; and mixtures thereof.

8. The composition according to claim 1, wherein the non-fatty alcohol surfactant different from the short chain surfactant is selected from the group consisting of glyceryl esters of fatty acids, polyethylene glycol esters of fatty acids, fatty acid esters of glucose, fatty acid esters of alkylglucose, oxyethylenated ethers of a fatty acid and of glucose, oxyethylenated ethers of a fatty acid and of alkylglucose, alkylpolyglucosides, phospholipids, and mixtures thereof.

9. The composition according to claim 1, wherein the composition further comprises at least one of a non-polar emollient and a polar emollient.

10. The composition according to claim 1, wherein the hydroxyl-containing acid is present in an amount of from 0.5 wt. % to 30 wt. %, relative to the total weight of the composition.

11. The composition according to claim 1, wherein the short chain surfactant is present in an amount ranging of from 0.1 wt. % to 15 wt. %, relative to the total weight of the composition.

12. The composition according to claim 1, wherein the at least two fatty alcohols are present in an amount ranging from 0.1 wt. % to 10 wt. %, relative to the total weight of the composition.

13. The composition according to claim 1, wherein the non-fatty alcohol surfactant different from the short chain surfactant is present in an amount ranging from 0.05 wt. % to 10 wt. %, relative to the total weight of the composition.

14. The composition according to claim 9, wherein the at least one of a non-polar emollient and polar emollient is present in an amount of from 0.1 wt. % to 20 wt. %, relative to the total weight of the composition.

15. A composition for caring keratin material, comprising:
(a) a keratolytic system, comprising:
(a1) at least one hydroxyl-containing acid, selected from the group consisting of α-hydroxyl acids, gluconic acid, lactobionic acid, salicylic acid, precursors thereof, and mixtures thereof, and
(a2) at least one PEG/PG-derived short chain surfactant,
(b) a liquid crystal system, comprising:
(b1) at least two fatty alcohols having 20 or more carbon atoms, and
(b2) at least one non-fatty alcohol surfactant different from the short chain surfactant, and
(c) optionally, at least one emollient,
wherein the composition does not comprise any fatty alcohols having less than 20 carbon atoms.

16. The composition according to claim 15, wherein the hydroxyl-containing acid is present in an amount of from 0.5 wt. % to 20 wt. %, relative to the total weight of the composition, the PEG/PG-derived short chain surfactant is present in an amount of from 0.1 wt. % to 10 wt. %, relative to the total weight of the composition, and/or the emollient is present and comprises a non-polar emollient and a polar emollient and is present in an amount of from 1 wt. % to 20 wt. %, relative to the total weight of the composition.

17. A method for improving the stability of a composition comprising a keratolytic system and optionally an emollient, comprising adding a liquid crystal system to the composition,
wherein the keratolytic system comprises at least one hydroxyl-containing acid, selected from the group consisting of α-hydroxyl acids, gluconic acid, lactobionic acid, salicylic acid, precursors thereof, and at least one PEG/PG-derived short chain surfactant, and
wherein the liquid crystal system comprises at least two fatty alcohols having 20 or more carbon atoms, and at least one non-fatty alcohol surfactant different from the short chain surfactant.

18. The method according to claim 17, further comprising adding an emollient, wherein the hydroxyl-containing acid is present in an amount of from 0.5 wt. % to 20 wt. %, relative to the total weight of the composition, and the emollient comprises a non-polar emollient and a polar emollient and is present in an amount of from 1 wt. % to 20 wt. %, relative to the total weight of the composition.
